(19) United States
(12) Patent Application Publication
(10) Pub. No.: US 2005/0159598 A1
(43) Pub. Date: Jul. 21, 2005

(54) Process for preparation of chemical compounds

(76) Inventors: Kenneth Ingold, Research Triangle Park, NC (US); Bing Liu, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/505,337

(22) PCT Filed: Feb. 25, 2003
(86) PCT No.: PCT/US03/05723

Related U.S. Application Data
(60) Provisional application No. 60/360,432, filed on Feb. 28, 2002.

(51) Int. Cl. C07D 417/02; C07D 413/02; C07F 1/02; C07F 7/04

(52) U.S. Cl. 540/603; 548/200; 548/215; 546/209; 260/665 R; 556/427

(57) ABSTRACT

The present invention relates to a process for the preparation of certain chemical compounds. In particular, the present invention relates to a method for the preparation of compounds that have been shown to activate human peroxisome proliferator activated receptors (“hPPARs”). The present invention also relates to certain chemical compounds useful as intermediates in the preparation of hPPAR active compounds.

The present invention relates to a process for the preparation of certain chemical compounds. In particular, the present invention relates to a method for the preparation of compounds that have been shown to activate human peroxisome proliferator activated receptors (“hPPARs”). The present invention also relates to certain chemical compounds useful as intermediates in the preparation of hPPAR active compounds.

### BACKGROUND

Several independent risk factors have been associated with cardiovascular disease. These include hypertension, increased fibrinogen levels, high levels of triglycerides, elevated LDL cholesterol, elevated total cholesterol, and low levels of HDL cholesterol. HMG CoA reductase inhibitors (“statins”) are useful for treating conditions characterized by high LDL-c levels. It has been shown that lowering LDL-c is not sufficient for reducing the risk of cardiovascular disease in some patients, particularly those with normal LDL-c levels. This population pool is identified by the independent risk factor of low HDL-c. The increased risk of cardiovascular disease associated with low HDL-c levels has not yet been successfully addressed by drug therapy (i.e. currently there are no drugs on the market that are useful for raising HDL-c). (Bisgaier, C. L.; Pape, M. E. Curr. Pharm. Des. 1998, 4, 53-70).

Syndrome X (including metabolic syndrome) is loosely defined as a collection of abnormalities including hyperinsulemia, obesity, elevated levels of triglycerides, uric acid, fibrinogen, small dense LDL particles, and plasminogen activator inhibitor 1 (PAI-1), and decreased levels of HDL-c.

NIDDM is described as insulin resistance, which in turn causes anomalous glucose output and a decrease in glucose uptake, by skeletal muscle. These factors eventually lead to impaired glucose tolerance (IGT) and hyperinsulinemia.

Peroxisome Proliferator Activated Receptors (PPARs) are orphan receptors belonging to the steroid/retinoid receptor superfamily of ligand-activated transcription factors. See, for example Willson T. M. and Wahli, W., Curr. Opin. Chem. Biol., 1, pp235-241 (1997) and Willson T. M. et. al., J. Med. Chem., 43, p527-549 (2000). The binding of agonist ligands to the receptor results in changes in the expression level of MRNA's encoded by PPAR target genes.

Three mammalian Peroxisome Proliferator-Activated Receptors have been isolated and termed PPAR-alpha, PPAR-gamma, and PPAR-delta (also known as NUC1 or PPAR-beta). These PPARs regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signalling cascade and lipid homeostasis (H. Keller and W. Wahli, Trends Endocrinol. Metab 291-296, 4 (1993)).

It has now been reported that thiazolidinediones are potent and selective activators of PPAR-gamma and bind directly to the PPAR-gamma receptor (J. M. Lehmann et. al., J. Biol. Chem. 12953-12956, 270 (1995)), providing evidence that PPAR-gamma is a possible target for the therapeutic actions of the thiazolidinediones.

Activators of the nuclear receptor PPARγ, for example troglitazone, have been shown in the clinic to enhance insulin-action, reduce serum glucose and have small but significant effects on reducing serum triglyceride levels in patients with Type 2 diabetes. See, for example, D. E. Kelly et al., Curr. Opin. Endocrinol. Diabetes, 90-96, 5 (2), (1998); M. D. Johnson et al., Ann. Pharmacother., 337-348, 32 (3), (1997); and M. Leutenegger et al., Curr. Ther. Res., 403-416, 58 (7), (1997).

The mechanism for this triglyceride lowering effect appears to be predominantly increased clearance of very low density lipoproteins (VLDL) through induction of lipoprotein lipase (LPL) gene expression. See, for example, B. Staels et al., Arterioscler. Thromb., Vasc. Biol., 1756-1764, 17 (9), (1997).

Fibrates are a class of drugs which may lower serum triglycerides 20-50%, lower LDLc 10-15%, shift the LDL particle size from the more atherogenic small dense to normal dense LDL, and increase HDLc 10-15%. Experimental evidence indicates that the effects of fibrates on serum lipids are mediated through activation of PPARα. See, for example, B. Staels et al., Curr. Pharm. Des., 1-14, 3 (1), (1997). Activation of PPARα results in transcription of enzymes that increase fatty acid catabolism and decrease de-novo fatty acid synthesis in the liver resulting in decreased triglyceride synthesis and VLDL production/secretion. In addition, PPARα activation decreases production of apoC-III. Reduction in apoC-III, an inhibitor of LPL activity, increases clearance of VLDL. See, for example, J. Auwerx et al., Atherosclerosis, (Shannon, Irel.), S29-S37,124 (Suppl), (1996).

Certain compounds that activate or otherwise interact with one or more of the PPARs have been implicated in the regulation of triglyceride and cholesterol levels in animal models. See, for example, U.S. Pat. No. 5,847,008 (Doebber et al.) and U.S. Pat. No. 5,859,051 (Adams et al.) and PCT publications WO 97/28149 (Leibowitz et al.) and WO99/04815 (Shimokawa et al.). In a recent report (Berger et al., J. Biol. Chem. 1999), vol. 274, pp. 6718-6725) it was stated that PPARδ activation does not appear to modulate glucose or triglyceride levels.

### BRIEF DESCRIPTION

The present invention relates to a process for the preparation of a compound of formula (IV): 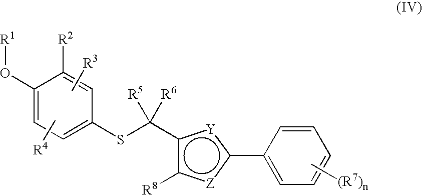
wherein,
R¹ is selected from the group consisting of H, —Si(R⁹)₃, —C(R¹⁰R¹⁰)C(O)₂H, benzyl, allyl, and C₁₋₆alkyl;
R², R³, and R⁴ are independently selected from the group consisting of H, C₁₋₃alkyl, —OCH₃, —CF₃, allyl, and halogen;
R⁵ and R⁶ are independently selected from the group consisting of H, phenyl, benzyl, C₁₋₆-alkyl, and allyl;
each R⁷ is independently selected from —CF₃, C₁₋₃alkyl, —OCH₃, or halogen;
R⁸ is selected from the group consisting of H, —CF₃, and C₁₋₆alkyl;
one of Y and Z is N and the other is S or O;
each R⁹ is independently selected from C₁₋₆alkyl, or arylC₁₋₆alkyl, or two R⁹ groups together with the silicon atom to which they are attached form a 5-7 membered ring;
each R¹⁰ is independently selected from H or C₁₋₃alkyl, or both R¹⁰ groups together with the carbon atom to which they are attached form a 3-6 membered ring; and
n=0, 1, 2, 3, 4, or 5;
comprising the steps of:
a) treating of a compound of formula (I) with an alkyl lithium reagent, magnesium (0), or magnesium (0) followed by treating with a dihalo zinc (II) reagent, 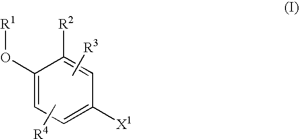
   wherein,
R¹, R², R³, and R⁴ are as defined above; and
X¹ is selected from the group consisting of Cl, Br, and I;
b) followed by treating with sulfur; and
c) followed by treating with a compound of formula (III), 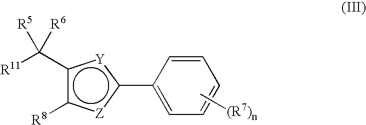
   wherein,
R⁵, R⁶, R⁷, R⁸, Y, Z, and n are as defined above;
R¹¹ is Cl, Br, I, or —OS(O)₂R¹²; and
R¹² is selected from the group consisting of C₁₋₆alkyl, C₆₋₁₀aryl, C₆₋₁₀arylC₁₋₆alkyl, and —CF₃.

Compounds of formula (IV) may be used as intermediates in the preparation of compounds that may activate human peroxisome proliferator activated receptors (hPPARs). Such compounds were disclosed in GB/0031107.6, filed Dec. 20, 2000.

### DETAILED DESCRIPTION OF THE INVENTION

In the present process, the intermediate compounds may be isolated between steps (a) and (b) and/or (b) and (c).

In a preferred aspect of the invention, intermediate compounds are not isolated between steps (a) and (b) or (b) and (c).

In a preferred aspect of the invention is a process in which the compound of formula (I) is treated with an alkyl lithium reagent.

More preferred is a process for the preparation of a compound of formula (IV), wherein R¹ is —Si(R⁹)₃, wherein each R⁹ is independently selected from C₁₋₆alkyl.

Even more preferred is a process for the preparation of a compound of formula (IV), wherein R¹ is —Si(CH₃)₂t-Bu.

Most preferred is a process for the preparation of a compound of formula (IV), wherein R¹ is —C(R¹⁰R¹⁰)C(O)₂H, and each R¹⁰ is —CH₃.

More preferred is a process for the preparation of a compound of formula (IV), wherein R² is —CH₃.

More preferred is a process for the preparation of a compound of formula (IV), wherein R³ and R⁴ are hydrogen.

More preferred is a process for the preparation of a compound of formula (IV), wherein R⁵ and R⁶ are hydrogen.

More preferred is a process for the preparation of a compound of formula (IV), wherein n is 2, one R⁷ is fluorine and the other is —CF₃.

Most preferred is a process for the preparation of a compound of formula (IV), wherein n is 2, one R⁷ is fluorine in the ortho position and the other is —CF₃ in the para position.

More preferred is a process for the preparation of a compound of formula (IV), wherein R⁸ is —CH₃.

More preferred is a process for the preparation of a compound of formula (IV), wherein Y is S, and Z is N.

More preferred is a process for the preparation of a compound of formula (IV), wherein R¹⁰ is —CH₃.

More preferred is a process for the preparation of compounds of formula (IV), wherein R¹¹ is Cl or —OS(O)₂R¹², and R¹² is C₁₋₆alkyl.

More preferred is a process for the preparation of a compound of formula (IV), wherein X¹ is Br.

In another aspect of the present invention are compounds of formula (IV) wherein:
R¹ is selected from the group consisting of —Si(R⁹)₃;
R², R³, and R⁴ are independently selected from the group consisting of H. C₁₋₃alkyl, —OCH₃, —CF₃, allyl, and halogen;
R⁵ and R⁶ are independently selected from the group consisting of H, phenyl, benzyl, C₁₋₆alkyl, and allyl;
each R⁷ is independently selected from —CF₃, C₁₋₃alkyl, —OCH₃, or halogen;
R⁸ is selected from the group consisting of H, —CF₃, and C₁₋₆alkyl;
one of Y and Z is N and the other is S or O;
each R⁹ is C₁-alkyl, or arylC₁₋₆alkyl, or two R⁹ groups together with the silicon atom to which they are attached form a 5-7 membered ring; and
n=0, 1, 2, 3, 4, or 5.

More preferred are compounds of formula (IV), wherein R¹ is —Si(CH₃)₂t-Bu.

More preferred are compounds of formula (IV), wherein R² is —CH₃

More preferred are compounds of formula (IV), wherein R³, R⁴, R⁵, and R⁶ are hydrogen.

More preferred are compounds of formula (IV), wherein n is 2, and one R⁷ is fluorine and the other is —CF₃.

Most preferred are compounds of formula (IV), wherein n is 2, one R⁷ is fluorine in the ortho position and the other is —CF₃ in the para position.

More preferred are compounds of formula (IV), wherein R⁸ is —CH₃.

More preferred are compounds of formula (IV), wherein Y is S, and Z is N.

In another aspect of the invention is featured a process for the preparation of compounds of formula (III), said process comprising the step of treating a compound of formula (XVII) with thioacetic acid, 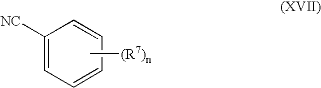
wherein:
each R⁷ is independently selected from —CF₃, C₁₋₃alkyl, —OCH₃, or halogen; and
n=0, 1, 2, 3, 4, or 5.

In another aspect of the present invention is featured a process for the preparation of a compound of formula (III), said process comprising the steps of:
a) treating a compound of formula (XVII) with thioacetic acid; followed by
b) treating with an α-halo-β-ketoester.

In another aspect of the present invention is featured a process for the preparation of a compound of formula (III), said process comprising the steps of:
a) treating a compound of formula (XVII) with thioacetic acid; followed by
b) treating with an α-halo-β-ketoester; and
c) treating with reducing agent.

In another aspect of the present invention are featured compounds of formula (V), 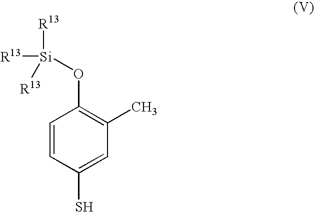
wherein:
R¹³ is C₁₋₆alkyl, or arylC₁₋₆alkyl, or two R⁹ groups together with the silicon atom to which they are attached form a 5-7 membered ring.

Another aspect of the present invention features a process for the preparation of compounds of formula (IV), wherein R¹ is —H, and R², R³, R⁴, R⁵, R⁶, R⁷, R⁸, Y, Z, and n are as defined above, said process further comprising the step of treating a compound of formula (IV), wherein R¹ is —Si(CH₃)₂t-Bu, with a base.

Another aspect of the present invention features a process for the preparation of compounds of formula (IV), wherein R¹ is —C(R¹⁰R¹⁰)C(O)₂H, R¹⁰ is —CH₃, and R², R³, R⁴, R⁵, R⁶, R⁷, R⁸, Y, Z, and n are as defined above, said method further comprising the steps of:
d) treating a compound of formula (IV), wherein R¹ is —Si(CH₃)₂t-Bu, with a base; and
e) treating with an alkylating agent.

Another aspect of the invention features a process for the preparation of compounds of formula (IV), wherein R¹ is —C(R¹⁰R¹⁰)C(O)₂H, R¹⁰ is —CH₃, and R², R³, R⁴, R⁵, R⁶, R⁷, R⁸, Y, Z, and n are as defined above, said method further comprising the steps of:
d) treating a compound of formula (IV), wherein R¹ is —Si(CH₃)₂t-Bu, with a base; and
e) treating with 1,1,1-trichloro-2-methylpropan-2-ol.

The compounds according to the invention may contain one or more asymmetric atoms and thus occur as racemates, racemic mixtures, single enantiomers, diastereomeric mixtures and individual diastereoisomers. All such isomeric forms of these compounds are expressly included in the present invention. Each stereogenic atom may be of the R or S configuration. Although the specific compounds exemplified in this application may be depicted in a particular stereochemical configuration, compounds having either the opposite stereochemistry at any given chiral center or mixtures thereof are also envisioned. When a compound of formula (IV) is desired as a single enantiomer, it may be obtained either by resolution of the final product or by stereospecific synthesis using methods known to those skilled in the art. See, for example, Stereochemistry of Organic Compounds by E. L. Eliel and S. H. Wilen (Wiley Interscience, 1994).

The terms “C₁₋₃alkyl” and “C₁₋₆-alkyl,” alone or in combination with any other term, refers to a straight-chain or branched-chain saturated aliphatic hydrocarbon radical containing the specified number of carbon atoms. Examples of alkyl radicals include, but are not limited to, methyl, ethyl, n-propyl, isopropyl, n-butyl, isobutyl, sec-butyl, tert-butyl, pentyl, isoamyl, n-hexyl and the like.

The term “C₆₋₁₄aryl” alone or in combination with any other term, refers to a carbocyclic aromatic moiety (such as phenyl or naphthyl) containing the specified number of carbon atoms, preferably from 6-14 carbon atoms, and more preferably from 6-10 carbon atoms. Examples of aryl radicals include, but are not limited to phenyl, naphthyl, indenyl, indanyl, azulenyl, fluorenyl, anthracenyl and the like.

The term “halogen” refers to fluorine, chlorine, bromine or iodine.

As used in herein, the term “alkyl lithium reagent” refers to a chemical compound that consists of an anionic C₁₋₆-alkyl portion and a corresponding lithium cation. Examples of such alkyl lithium reagents are n-butyl lithium, sec-butyl lithium, and tert-butyl lithium. Such alkyl lithium reagents are commercially available, conveniently as solutions in an appropriate solvent such as hexanes or cyclohexane, or may be prepared by methods known to those skilled in the art.

As used herein, the term “sulfur” refers to elemental sulfur.

As used herein, the term “intermediate compounds” refers to chemical compounds that are products of steps that comprise a chemical process. Such intermediates may or may not be amenable to chemical isolation, depending on their structure, chemical stability, or chemical reactivity.

As used herein, the term “ortho position” refers to the position on an aryl ring that is disposed in a 1,2-orientation relative to another substituent on said ring. For example, in the compound 1-chloro-2-methyl benzene the methyl group is oriented ortho to the chloro substituent.

As used herein, the term “para position” refers to the position on an aryl ring that is disposed in a 1,4-orientation relative to another substituent on said ring. For example, in the compound 1-chloro-4-methyl benzene the methyl group is oriented para to the chloro substituent.

As used herein, the term “base” refers to chemical compounds known to those skilled in the art as either Bronsted or Lewis bases. Examples of such bases known to those skilled in the art include lithium hydroxide, sodium hydroxide, potassium hydroxide, trialkylamines such as triethylamine, and tetraalkyl ammonium halides such as tetra-n-butylammonium fluoride. In addition, included are compounds known to those skilled in the art to afford aqueous solutions that are basic in nature. Examples of such compounds are sodium carbonate, sodium bicarbonate, potassium carbonate, and potassium bicarbonate.

As used herein, the term “α-halo-β-ketoester” refers to a compound of formula (XXII), 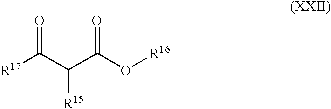
wherein:
R¹⁵ is halogen;
R¹⁶ is C₁₋₆alkyl, C₃₋₈cycloalkyl, C₆₋₁₄aryl, or C₆₋₁₄arylC₁₋₆alkyl; and
R¹⁷ is hydrogen, —CF₃, or C₁₋₆alkyl.

As used herein, the term “reducing agent” refers to a reagent or combination of reagents known to those skilled in the art capable of reducing an α-halo-β-ketoester. Among these reagents or combination of reagents are lithium aluminum hydride, borane, and di-isobutylaluminum hydride (DIBAL).

As used herein, the term “magnesium (0)” refers to elemental magnesium in a form known to those skilled in the art to be useful in preparing so-called “Grignard reagents.” Among such magnesium (0) forms are magnesium turnings and activated magnesium (0), so called “Rieke magnesium.”

As used herein, the term “dihalo zinc (II) reagent” refers to a compound containing two halogen atoms and zinc in the (II) oxidation state. Among such reagents known to those skilled in the art to be useful in such processes are zinc (II) bromide, zinc (II) iodide, and zinc (II) chloride.

Compounds of formulae (I) and (Ill) may be prepared by methods known to those of skill in the art. The following synthetic schemes are meant to represent examples only and are not meant to limit the invention in any way. In all of the schemes described below, it is understood that protecting groups may be employed where necessary in accordance with general principles known to those skilled in the art, for example, see T. W. Green and P. G. M. Wuts (1991) Protecting Groups in Organic Synthesis, John Wiley & Sons. These groups may be removed at a convenient stage of the compound synthesis using methods known those of skill in the art. The selection of processes as well as the reaction conditions and order of their execution shall be consistent with the preparation of compounds of formulae (I) and (III). Those of skill in the art will recognize that if a stereocenter exists in compounds of Formulas (I) and (III), and (IV) the present invention is meant to include both enantiomers, mixtures of such enantiomers and the individual enantiomers substantially free of the opposite enantiomer. In addition, when a compound contains more than one stereocenter, one of skill in the art will recognize that the present invention is meant to include mixtures of diastereomeric compounds, mixtures of enantiomers and the individual enantiomers substantially free of the opposite enantiomer.

Compounds of formula (IV), 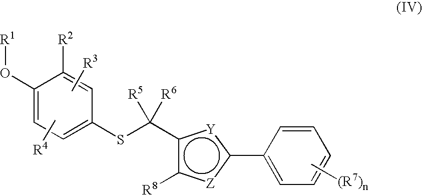
wherein,
R¹ is selected from the group consisting of H, —Si(R⁹)₃, —C(R¹⁰R¹⁰)C(O)₂H, benzyl, allyl, and —CH₃;
R², R³, and R⁴ are independently selected from the group consisting of H, C₁₋₃alkyl, —OCH₃, —CF₃, allyl, and halogen;
R⁵ and R⁶ are independently selected from the group consisting of H, phenyl, benzyl, C₁₋₆alkyl, and allyl;
each R⁷ is independently selected from —CF₃, C₁₋₃alkyl, —OCH₃, or halogen;
R⁸ is selected from the group consisting of H, —CF₃, and C₁₋₆-alkyl;
one of Y and Z is N and the other is S or O;
each R⁹ is independently selected from C₁₋₆alkyl, arylC₁₋₆alkyl, or two R⁹ groups together with the silicon atom to which they are attached form a 5-7 membered ring;
each R¹⁰ is independently selected from H or C₁₋₃alkyl, or both R¹⁰ groups together with the carbon atom to which they are attached form a 3-6 membered ring, and at least one R⁹ group must be other than H; and
n=0, 1, 2, 3, 4, or 5;
are prepared by a process in which a compound of formula (I) is treated with an alkyl lithium reagent, magnesium (0), or magnesium (0) followed by treating with a dihalo zinc (II) reagent, 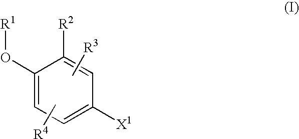
   wherein,
R¹, R², R³, and R⁴ are as defined above; and
X¹ is selected from the group consisting of Cl, Br, and I;
followed by treatment with sulfur, and then followed by treatment with a compound of formula (III), 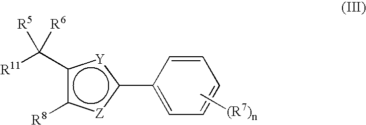
   wherein,
R⁵, R⁶, R⁷, R⁸, Y, Z, and n are as defined above;
R¹¹ is Cl, Br, I, or —OS(O)₂R¹²; and
R¹² is selected from the group consisting of C₁₋₆alkyl, C₆₋₁₀aryl, and C₆₋₁₀arylC₁₋₆alkyl, and —CF₃.

These reactions may be performed in a manner in which intermediate compounds are isolated before using them in the next appropriate chemical step. For example, a compound of formula (I), wherein R¹ is —Si(R⁹)₃, and R², R³, R⁴, X¹, and R⁹ are as hereinbefore defined, may be treated with an alkyl lithium reagent to affect what is known to those skilled in the art as a halogen-metal exchange reaction, followed by treatment with sulfur and isolation of the intermediate product of formula (V), 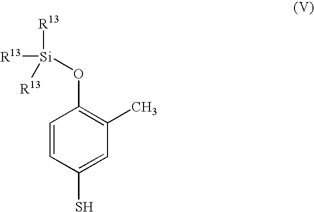
wherein:
R¹³ is C₁₋₆-alkyl, or arylC₁₋₆alkyl, or two R⁹ groups together with the silicon atom to which they are attached form a 5-7 membered ring.

These reactions are typically performed in an aprotic solvent, such as tetrahydrofuran (THF) or preferably methyl tert-butyl ether (MTBE), and at a temperature from −78° C. to 0° C., preferably −30° C. Further, the alkyl lithium reagent may be one known to those skilled in the art capable of effecting a halogen-metal exchange reaction, such as sec-butyl lithium, tert-butyl lithium, or preferably n-butyl lithium. Such alkyl lithium reagents are commercially available, conveniently in an appropriate solvent such as hexanes or cyclohexanes, or may be prepared by methods known to those skilled in the art.

The compound of formula (V) may then be treated with a base to deprotonate the thiol to form a thiolate anion, followed by treatment with a compound of formula (III), to afford a compound of formula (IV).

Alternatively, compounds of formula (IV) may be prepared by a process in which a compound of formula (I) is treated with an appropriate alkyl lithium reagent, n-butyl lithium for example, in an appropriate solvent, MTBE for example, at a temperature from −78° C. to 0° C., preferably −30° C., followed by treatment with sulfur, and then followed by treatment with a compound of formula (III). In this embodiment, the compound of formula (V) is not isolated, but instead the desired thiolate is generated in situ and is allowed to react with a compound of formula (III) to afford a compound of formula (IV). These reactions are typically performed by adding an appropriate alkyl lithium reagent to the compound of formula (I) to affect a halogen-metal exchange reaction, followed by the addition of sulfur to the reaction mixture, and finally adding the resulting thiolate to a solution of a compound of formula (III). The reaction may also be performed in the reverse manner in which the compound of formula (III) is added to a solution of the in situ generated thiolate. For example, as shown in Scheme I, (4-bromo-2-methylphenoxy)(tert-butyl)dimethylsilane was allowed to react with n-butyl lithium (n-BuLi), followed by treatment with sulfur to afford a compound of formula (VI). In a separate reaction vessel, [2-(2-fluoro-4-methylphenyl)4-methyl-1,3-thiazol-5-yl]methanol (VII) was allowed to react with methanesulfonyl chloride in the presence of triethylamine to afford a compound of formula (VIII), wherein LG is —Cl or —OS(O)₂CH₃ or a mixture thereof. The solution of compound (VI) was then added to a solution of compound (VIII) to afford 5-{[(4-{[tert-butyl(dimethyl)silyl]oxy}-3-methylphenyl)thio]methyl)2-[2-fluoro4-(trifluoromethyl)phenyl]4-methyl-1,3-thiazole (IX). The thiolate structure shown for intermediate compound (VI) is presented only as an example and is not meant to limit the scope of the present invention in any way.

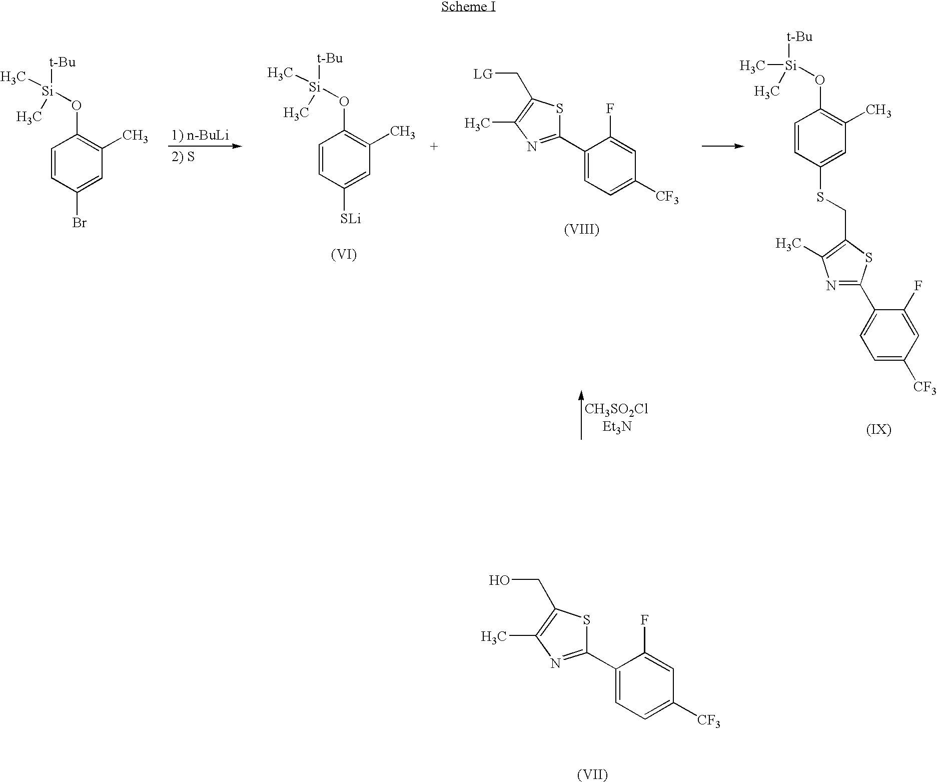

Alternatively, compounds of formula (IV) may be prepared by a process in which a compound of formula (I) is treated with an appropriate magnesium (0) reagent, in an appropriate solvent, THF or MTBE for example, at a temperature from ambient to 50° C., followed by treatment with sulfur, and then followed by treatment with a compound of formula (III). In this embodiment, the compound of formula (V) is not isolated, but instead the desired thiolate is generated in situ and is allowed to react with a compound of formula (III) to afford a compound of formula (IV).

Alternatively, compounds of formula (IV) may be prepared by a process in which a compound of formula (I) is treated with an appropriate magnesium (0) reagent, in an appropriate solvent, MTBE for example, at a temperature from ambient to 50° C., followed by treating with a dihalo zinc (II) reagent, followed by treating with sulfur, and then followed by treating with a compound of formula (III). In this embodiment, the compound of formula (V) is not isolated, but instead the desired thiolate is generated in situ and is allowed to react with a compound of formula (III) to afford a compound of formula (IV).

The compounds of formula (I), wherein R¹ is —Si(R⁹)₃, and R², R³, R⁴, R⁹ and X¹ are as hereinbefore defined, are either commercially available or may be prepared from compounds of formula (I) wherein R¹ is H by methods known to those skilled in the art. These reactions are typically performed in an aprotic solvent, such as dichloromethane, chloroform, or preferably toluene, and in the presence of an appropriate trialkylsilyl trifluoromethanesulfonate or trialkylsilyl chloride, tert-butyldimethylsilyl chloride for example, and at a temperature from −30° C. to 30° C., preferably 10-15° C. In addition, the reaction may be performed in the presence of a catalyst, for example 4-dimethylaminopyridine (DMAP).

Compounds of formula (I), wherein R¹ is —Si(R⁹)₃, R², R³, R⁴, R⁹ and X¹ are as hereinbefore defined, are either commercially available or may be prepared from compounds of formula (I) wherein R¹ and X¹ are H by methods known to those skilled in the art. The silylation reactions are typically performed in an aprotic solvent, such as dichloromethane, chloroform, or preferably toluene, and in the presence of an appropriate trialkylsilyl chloride, tert-butyldimethylsilyl chloride for example, and at a temperature from −30° C. to 30° C., preferably 10-15° C. In addition, the reaction may be performed in the presence of a catalyst, for example 4-dimethylaminopyridine (DMAP). The halogenation reactions are typically performed in an aprotic solvent, acetonitrile for example, at a temperature from 0° C. to 50° C., preferably ambient temperature, and in the presence of a compound capable of halogenating the benzene ring, N-bromosuccinimide, for example. For example, as shown in Scheme II, a solution of o-cresol in toluene, and in the presence of DMAP, was allowed to react with t-butyldimethylsilyl chloride to afford compound (X). Subsequently, compound (X) was allowed to react with NBS in acetonitrile to afford (4-bromo-2-methylphenoxy)(tert-butyl)dimethylsilane (XI). 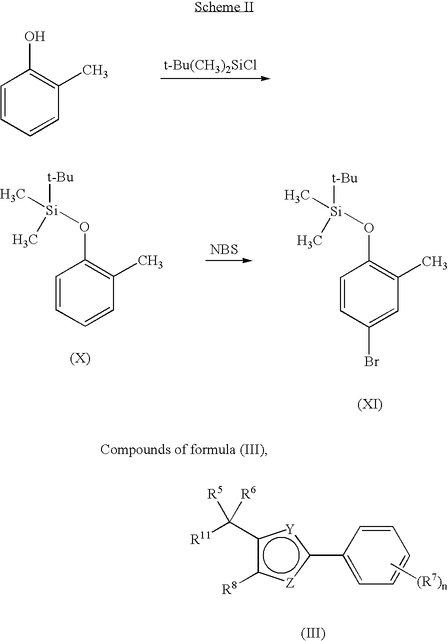
wherein,
R⁵, R⁶, R⁷, R⁸, Y, Z, and n are as defined above;
R¹¹ is Cl, Br, I, or —OS(O)₂R¹²; and
R¹² is selected from the group consisting of C₁₋₆alkyl, C₆₋₁₀aryl, C₆₋₁₀arylC₁₋₆alkyl, and —CF₃;
may be prepared from compounds of formula (III), wherein R¹¹ is —OH, and R⁵, R⁶, R⁷, R⁸, Y, Z, and n are as defined above, by reaction with a reagent, or combination of reagents, capable of converting the hydroxy group into a leaving group, such as a halide or alkyl or aryl sulfonyl group. These reactions are typically performed in an aprotic solvent such as dichloromethane, chloroform, acetonitrile, MTBE, or preferably a mixture of acetonitrile and MBTE, in the presence of a base, triethylamine for example, and at a temperature from −78° C. to 25° C., preferably −20 to −15° C. Among the reagents or combination of reagents that are capable of converting the hydroxy group to a leaving group are p-toleuensulfonyl chloride, or preferably methanesulfonyl chloride, in the presence of a base such as pyridine, DMAP, or preferably triethylamine. For example, {2-[2-fluoro-4-(trifluoromethyl)phenyl]4-methyl-1,3-thiazol-5-yl}methanol (XIII) was allowed to react with methanesulfonyl chloride in a mixture of acetonitrile and MTBE and in the presence of triethylamine at a temperature of −15 to −20° C. to afford either 5-(chloromethyl)-2-[2-fluoro-4-(trifluoromethyl)phenyl]-4-methyl-1,3-thiazole (XIV) or {2-[2-fluoro-4-(trifluoromethyl)phenyl]-4-methyl-1,3-thiazol-5-yl}methyl methanesulfonate (XV), or a mixture of both.

Compounds of formula (III), wherein R¹¹ is —OH, and R⁵, R⁶, R⁷, R⁸, Y, Z, and n are as defined above, may be prepared from compounds of formula (XVI), 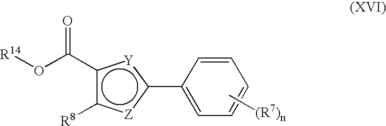
wherein R⁷, R⁸, Y, Z, and n are as hereinbefore defined and R¹⁴ is C₁₋₆-alkyl, by reaction with a reagent or combination of reagents known to those skilled in the art capable of reducing an ester to an alcohol or capable of addition to an ester. Among such reagents known to those skilled in the art are lithium aluminum hydride (LAH), alkyl lithium reagents, or alkyl magnesium halides (so-called “Grignard” reagents). These reactions are typically performed in an aprotic solvent such as THF and at a temperature from −78 to 0° C., preferably −10 to −15° C. For example, a THF solution of ethyl 2-[2-fluoro-4-(trifluoromethyl)phenyl]-4-methyl-1,3-thiazole-5-carboxylate (XVII) was allowed to react with LAH at −10 to −15° C. to afford (2-[2-fluoro-4-(trifluoromethyl)phenyl]-4-methyl-1,3-thiazol-5-yl}methanol (XIII).

Compounds of formula (XVI), wherein R⁷, R⁸, Y, Z, and n are as hereinbefore defined and R¹⁴ is C₁₋₆alkyl, may be prepared from compounds of formula (XVII), 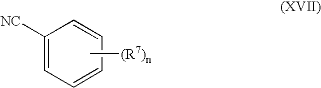
wherein R⁷ and n are as hereinbefore defined. Compounds of formula (XVII) may be allowed to react with a suitable sulfur donor, thioacetic acid for example, in the presence an appropriate Lewis acid, boron trifluoride etherate for example, in an aprotic solvent, toluene for example, and at a temperature from 0° C. to 50° C., preferably 20° C. See, J. Y. Gauthier, et al. Phosphorous, Sulfur, and Silicon, 1994, Vol. 95-96, pp. 325-326. For example, as shown in Scheme III, 2-fluoro-4-(trifluoromethyl)benzonitrile was allowed to react with thioacetic acid in toluene and in the presence of boron trifluoride etherate to afford 2-fluoro4-(trifluoromethyl)benzenecarbothioamide (XVIII). Compound (XVIII) may then be allowed to react with and α-halo-β-keto ester, such as ethyl 2-chloroacetoacetate, in an aprotic solvent, toluene for example, and at a temperature of 75° C. to 125° C., preferably 100° C., to afford ethyl 2-[2-fluoro-4-(trifluoromethyl)phenyl]4-methyl-1,3-thiazole-5-carboxylate (XIX).

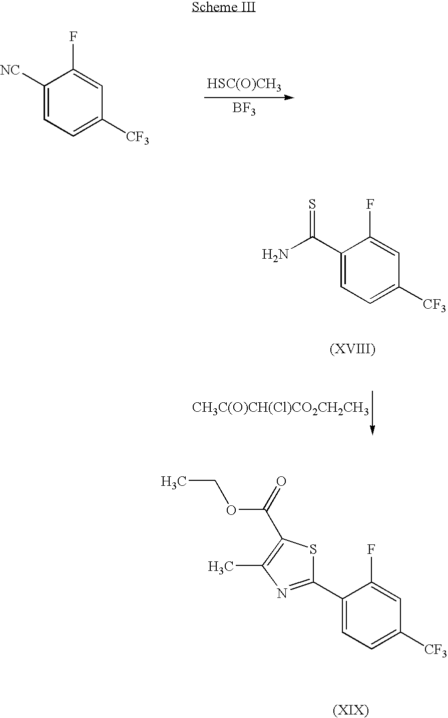

Compounds of formula (XVII) are either commercially available or may be prepared by methods known to those skilled in the art.

Compounds of formula (IV), wherein R¹ is H and R², R³, R⁴, R⁵, R⁶, R⁷, R⁸, Y, Z, and n are as hereinbefore defined may be prepared from compounds of formula (IV), wherein R¹ is —Si(R⁹)₃, and R², R³, R⁴, R⁵, R⁶, R⁷, R⁸, R⁹, Y, Z, and n are as hereinbefore defined by reaction with a reagent or combination of reagents known to those skilled in the art capable of acting as either a Bronsted or Lewis base. Among the reagents known to those skilled in the art capable of acting as either Bronsted or Lewis base include lithium hydroxide, sodium hydroxide, potassium hydroxide, trialkylamines such as triethylamine, and tetraalkyl ammonium halides such as tetra-n-butylammonium fluoride. In addition, included are compounds known to those skilled in the art to afford aqueous solutions that are basic in nature. Examples of such compounds are sodium carbonate, sodium bicarbonate, potassium carbonate, and potassium bicarbonate. For example, as shown in Scheme IV, 5-{[(4-{[tert-butyl(dimethyl)silyl]oxy}-3-methylphenyl)thio]methyl}-2-[2-fluoro-4-(trifluoromethyl)phenyl]-4-methyl-1,3-thiazole (IX) was allowed to react with sodium hydroxide solution to afford 4-[({2-[2-fluoro-4-(trifluoromethyl)phenyl]-4-methyl-1,3-thiazol-5-yl}methyl)thio]-2-methylphenol (XX).

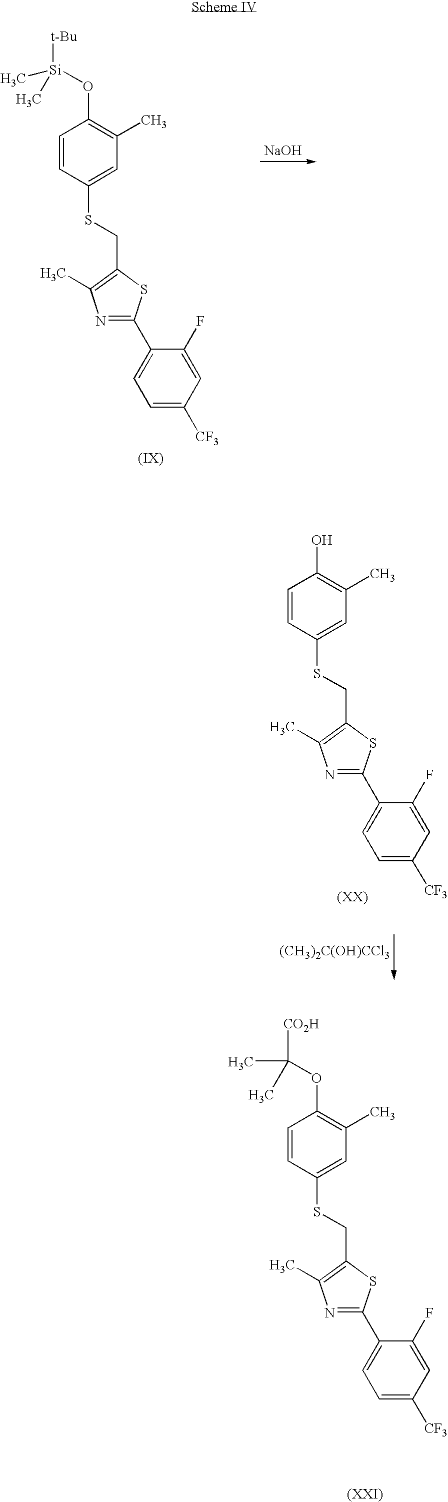

Compounds of formula (IV), wherein R¹ is —C(R¹⁰R¹⁰)C(O)₂H, and R², R³, R⁴, R⁵, R⁶, R⁷, R⁸, R¹⁰, Y, Z, and n are as hereinbefore defined may be prepared from compounds of formula (IV), wherein R¹ is —OH, and R², R³, R⁴, R⁵, R⁶, R⁷, R⁸, Y, Z, and n are as hereinbefore defined, by reaction with a reagent capable of alkylating the phenol to provide the desired product. Among the reagents capable of alkylating the phenol to provide the desired product is 1,1,1-trichloro-2-methyl-propanol. The alkylation reaction may be performed in a polar solvent, acetone for example, in the presence of a base, sodium hydroxide for example, and at a temperature of 0-50° C., preferably 36-38° C. For example, as shown in Scheme IV, 4-[({2-[2-fluoro-4-(trifluoromethyl)phenyl]-4-methyl-1,3-thiazol-5-yl}methyl)thio]-2-methylphenol (XX) was allowed to react with 1,1,1-trichloro-2-methyl-propanol in acetone and in the presence of sodium hydroxide to afford 2-{4-[({2-[2-fluoro-4-(trifluoromethyl)phenyl]-4-methyl-1,3-thiazol-5-yl}methyl)thio]-2-methylphenoxy}-2-methylpropanoic acid (XXI).

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| i. v. (intravenous); | Hz (Hertz); |
| MHz (megahertz); | mol (moles); |
| mmol (millimoles); | rt (room temperature); |
| min (minutes); | h (hours); |
| mp (melting point); | TLC (thin layer chromatography); |
| Tᵣ (retention time); | RP (reverse phase); |
| MeOH (methanol); | i-PrOH (isopropanol); |
| TEA (triethylamine); | TFA (trifluoroacetic acid); |
| TFAA (trifluoroacetic anhydride); | THF (tetrahydrofuran); |
| DMSO (dimethylsulfoxide); | AcOEt (ethyl acetate); |
| DME (1,2-dimethoxyethane); | DCM (dichloromethane); |
| DCE (dichloroethane); | DMF (N,N- |
| | dimethylformamide); |
| DMPU (N,N′-dimethylpropyleneurea); | (CDI (1,1-carbonyldiimidazole); |
| IBCF (isobutyl chloroformate); | HOAc (acetic |
| | acid); |
| HOSu (N-hydroxysuccinimide); | HOBT (1-hydroxybenzotriazole); |
| mCPBA (meta-chloroperbenzoic acid; | EDC (ethylcarbodiimide |
| | hydrochloride); |
| BOC (tert-butyloxycarbonyl); | FMOC (9- |
| | fluorenylmethoxycarbonyl); |
| DCC (dicyclohexylcarbodiimide); | CBZ |
| | (benzyloxycarbonyl); |
| Ac (acetyl); | atm (atmosphere); |
| TMSE (2-(trimethylsilyl)ethyl); | TMS (trimethylsilyl); |
| TIPS (triisopropylsilyl); | TBS (t-butyldimethylsilyl); |
| DMAP (4-dimethylaminopyridine); | BSA (bovine serum albumin) |
| ATP (adenosine triphosphate); | HRP (horseradish |
| | peroxidase); |
| DMEM (Dulbecco's modified Eagle medium); | |
| HPLC (high pressure liquid chromatography); | |
| BOP (bis(2-oxo-3-oxazolidinyl)phosphinic chloride); | |
| TBAF (tetra-n-butylammonium fluoride); | |
| HBTU (O-Benzotriazole-1-yl-N,N,N′,N′-tetramethyluronium | |
| hexafluorophosphate). | |
| HEPES (4-(2-hydroxyethyl)-1-piperazine ethane sulfonic acid); | |
| DPPA (diphenylphosphoryl azide); | |
| fHNO₃ (fumed HNO₃); and | |
| EDTA (ethylenediaminetetraacetic acid). | |

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APIiii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

### Example 1

### Preparation of ethyl 2-[2-fluoro4-(trifluoromethyl)phenyl]-4-methyl-1,3-thiazole-5-carboxylate (XIX)

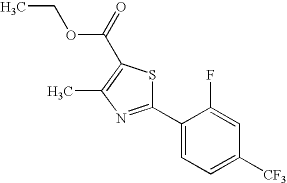

A reactor was charged with toluene (4 vol), thioacetic acid (1.0 vol), and boron trifluoride etherate (1.64 vol). A solution of 2-fluoro4-trifluoromethylbenzonitrile (1 eq, 1 wt) in toluene (1 vol) is added to the mixture via a pump over 60 minutes with reaction control at 20° C. After the addition is complete, the mixture is allowed to stir for 3 hours. The temperature is then brought to 5° C. Process water (1 vol) is added over 30 minutes to quench boron trifluoride with reaction control at 5° C. Once the quenching is complete, process water (3 vol) is added to dilute the reaction mixture. The aqueous layer is separated, and 10% ammonia solution (4 vol) is added over 30 minutes. The reaction is highly exothermic and active cooling is engaged (reaction control at 5° C.). CAUTION: The ammonia washing is separated from the toluene phase. The mixture is brought to 20° C. with reaction control at 20° C. The organic layer is washed with process water (2×4 vol) and concentrated under reduced pressure (90-60 mm Hg, 50° C.) to 3 vol. The mixture is used directly in the condensation with ethyl 2-chloroacetoacetate. Ethyl 2-chloroacetoacetate (1.1 eq, 0.8 vol) is added to the toluene solution and the mixture is heated at 100° C. (reaction control) until the reaction is complete (ca. 14 hours). The reaction mixture is cooled to 50° C. Toluene (2 vol) is removed under reduce pressure (90-60 mm Hg, 50° C.). The volume reduces to 3 vol. Ethanol (4 vol) is added and solvent (4 vol) is removed. During the concentration, batch temperature is maintained at 35-40° C. (jacket ca. 70 ° C.), vacuum at 140 mmHg. When the temperature gets lower than 28° C., product will precipitate out of the mixture. Ethanol (4 vol) is added and the volume reduces to 6 vol, followed by adding process water (0.3 vol). The mixture is allowed to cool to 20° C. over 1 hour and remain there for 1 h. The solid is collected by filtration, washed with cold aqueous ethanol prepared above, sucked to dryness, and dried under vacuum at 40° C. to a constant weight.

### Example 2

### Preparation of {2-[2-fluoro-4-(trifluoromethyl)phenyl]-4-methyl-1,3-thiazol-5-yl}methanol (XIII)

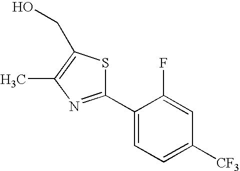

A reactor was charged with THF (4 vol) and 1 M LAH/THF (0.62 eq, 1.85 vol). The temperature is set in reaction control and brought to −15° C. Compound (XIX) (1 eq, 1 wt). is dissolved in THF (2 vol) and the solution is added using a metering pump over 1.5 hour while keeping the temperature between −10 and −15° C. After the addition is complete, the mixture is allowed to stir at that temperature for 0.5 h. In process check (IPC) confirms that the starting material was completely consumed. The reaction was then quenched by adding a mixture of process water and THF (1/1, 0.15 vol) over 30 minutes, 20% NaOH solution (0.056 vol) over 15 minutes, and process water (0.26 vol) over 15 minutes. During the quenching process, the internal temperature is kept at −10 to 15° C. and nitrogen is used to dilute generated hydrogen. After the addition, the mixture is stirred at 20° C. (reaction control) for 0.5 h. The granular residue is filtered and washed with THF (3×1 vol). The combined filtrate is concentrated to 2 vol (300 mmHg, reaction control set at 40° C.). Heptane (6 vol) is added, and the mixture is reduced to 6 vol. The mixture is allowed to ramp to 20° C. over 1 h and then chilled at 10° C. for 30 min. The solid is collected by vacuum filtration, washed with heptane (1 vol), dried at 50° C., 10-15 in Hg vac to a constant weight.

### Example 3

### Preparation of (4-bromo-2-methylphenoxy)(tert-butyl)dimethylsilane

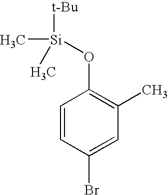

To a slurry of o-cresol (1 mol, 108 g) and DMAP (1.25 eq, 1.25 mol, 152 g) in toluene (0.43 L) is added tert-butyldimethylsilyl chloride (1.25 eq, 1.25 mol, 375 g of a 50% solution in toluene) at such a rate that the reaction temperature is maintained between 10 and 15° C. The thick slurry is stirred at rt for 5 h, then treated at rt with water (0.29 L). The resulting 2-phase system is stirred for 5 min, then treated with 1N hydrochloric acid (0.12L). After 5 min, the two clear, colorless layers are separated. The aqueous layer is removed and the organic layer is washed with water (0.29 L). The organic layer is washed with 1N sodium hydroxide (0.14 L) for 5 min and the layers separated. The organic layer is washed with water (0.15 L), then evaporated to give a nearly colorless oil.

Stage 2: The neat product of stage 1 (1.0 mol, 223 g) is added in a slow stream to a pre-heated (25° C.) slurry of N-bromosuccinimide (0.96 eq, 0.96 mol, 171 g) in acetonitrile (0.90 L) contained in a vessel protected from light and at such a rate that with rt water bath cooling, the reaction temperature is maintained at 20-25° C. The reaction typically takes 0.5-1.5 h for completion.

To the mixture is added heptane (0.67 L) and water (0.67 L) to produce two clear layers. The product-containing upper layer is separated and washed with water (0.67 L). The resulting organic layer is freed of solvent by rotary evaporator and the resulting oil is dissolved in toluene (0.20 L) followed by distillation of toluene. The drying process is repeated to produce a pale yellow oil (92-95%).

### Example 4

### Preparation of 5-{[(4-[tert-butyl(dimethyl)silyl]oxy}-3-methylphenyl)thio]methyl)2-[2-fluoro-4-(trifluoromethyl)phenyl]-4-methyl-1,3-thiazole (IX)

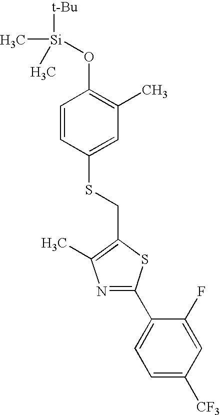

A solution of (4-bromo-2-methylphenoxy)(tert-butyl)dimethylsilane (417 g, 1.38 mol) and MTBE (1.80 L) was stirred and chilled to −30° C., then treated over 10 min with 2M n-BuLi/cyclohexanes (1.16 eq, 1.60 mol, 0.800 L). The solution was allowed to warm to 0° C. and maintained at that temperature until trans-metallation was deemed to be complete. The clear, pale yellow solution was cooled to −15° C., and sulfur (1.0 eq., 1.38 mol, 44.2 g) was added via solid-addition funnel at such a rate that the temperature was maintained between −15 and −10° C. The clear, light yellow solution was stirred at −15° C. until reaction was deemed to be complete (HPLC); then held briefly at −15° C.

During the running of the metallation phase, a separate reactor was charged with {2-[2-fluoro-4-(trifluoromethyl)phenyl]-4-methyl-1,3-thiazol-5-yl}methanol (XIII) (1.20 mol, 350 g), triethylamine (1.08 eq, 1.30 mol, 0.179 L), acetonitrile (1.20 L) and methyl t-butyl ether (0.80 L). The slurry was chilled to −20° C. and methanesulfonyl chloride (1.07 eq, 1.28 mol, 0.097 L) was added such that the reaction temperature was maintained between −20 and −15° C. The mixture was held at −15° C. until stage 1 was complete.

The MTBE solution of the metallation product was added via cannula to the acetonitrile/MTBE slurry of the product of (XII) such that the reaction temperature was maintained between −15 and −12° C. The resulting slurry was allowed to warm to 10° C. over 2.5 h and then was quenched with water (3 L). The layers were separated and the organic layer was washed with water (3 L). The organic layer was filtered through diatomaceous earth and the resulting clear filtrate was freed of a small residual layer of water. The organic solution was evaporated to approximately 1.1 L volume, then treated with ethanol (1. 1L) to initiate precipitation of product. The solid was filtered, and the resulting cake washed with ethanol (3×0.2 L) that was pre-cooled to 10° C. The resulting colorless product was dried in vacuo at 55° C., to a constant mass of 538 g (85%).

### Example 6

### Preparation of 4-[({2-[2-fluoro-4-(trifluoromethyl)phenyl]-4-methyl-1,3-thiazol-5-yl}methyl)thio]-2-methylphenol (XX)

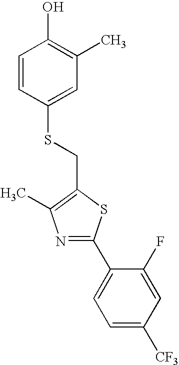

A slurry of 5-{[(4-{[tert-butyl(dimethyl)silyl]oxy}-3-methylphenyl)thio]methyl)2-[2-fluoro4-(trifluoromethyl)phenyl]-4-methyl-1,3-thiazole (IX) (1.00 mol, 527 g) in ethyl alcohol (1.85 L) was treated at ambient temperature with 5N NaOH (2 eq, 2.00 mol, 0.40 L). The resulting slurry was heated at 40° C. for 3 h, after which time heptane (1.2 L) and water (1 L) were added. The lower layer was separated, cooled to 15° C., then treated with 6N HCl (2.1 eq, 2.1 mol, 0.350 L) causing the product to precipitate. The product was filtered, washed with water (3×0.50 L) and heptane (2×0.40 L), and dried at 55° C. under vacuum to produce a colorless solid, 408.9 g (99%).

### Example 7

### Preparation of 2-{4-[({2-[2-fluoro-4-(trifluoromethyl)phenyl]-4-methyl-1,3-thiazol-5-yl}methyl)thio]-2-methylphenoxy}-2-methylpropanoic acid (XXI)

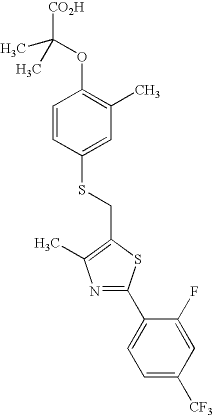

To a slurry of 20-40 mesh sodium hydroxide (8 eq ) and acetone (10 vol) was added 4-[({2-[2-fluoro-4-(trifluoromethyl)phenyl]-4-methyl-1,3-thiazol-5-yl}methyl)thio]-2-methylphenol (XX) (1 eq, 1 wt), and the resulting slurry was stirred at 32° C. for 3 h. A solution of 1,1,1-trichloro-2-methyl-propanol hydrate (1.7 eq) in acetone (5 vol) was added dropwise over 60 min during which the temperature was allowed to rise and was maintained between 36 and 38° C. The reaction mixture was allowed to cool to rt and the volume was reduced in vacuo to ¼-½ of the total volume. Methyl-t-butylether (10 vol) was added, and 1N hydrochloric acid (10 vol) was added at such a rate as to keep the temperature under 25° C. The organic layer was washed with water (2×4 vol), dried over sodium sulfate (0.1-0.5 wt), filtered, and concentrated to 5 volumes. The solution was heated to approximately 50° C. and heptane (3.5 vol) was added slowly. The solution was then heated to reflux temperature and additional heptane (3.5 vol) was added at such a rate as to maintain the temperature above 55° C. The solution was then distilled at atmospheric pressure to 7 volumes. The solution was cooled to 70° C. over 30 min, during which time the product begins to crystallize. The reaction mixture was then cooled to 20° C. over 30 min. Heptane (1 vol) was added and the slurry is stirred for 15 min at 20° C. The solid was filtered and washed with heptane (2 vol). The off-white solid was returned to the reactor and slurried in 5 vol of heptane. The slurry was heated to 60° C. over 20 min and held at 60° C. for 10 min. The slurry is cooled back to 15° C. over 30 min, and the resulting solid was collected by filtration, washed with heptane (1 vol) dried in vacuo at 50° C., to constant mass (511 g, 70%).

### Example 8

### Preparation of 2-{4-[({2-[2-fluoro-4-(trifluoromethyl)phenyl]-4-methyl-1,3-thiazol-5-yl}methyl)thio]-2-methylphenoxy}-2-methylpropanoic acid

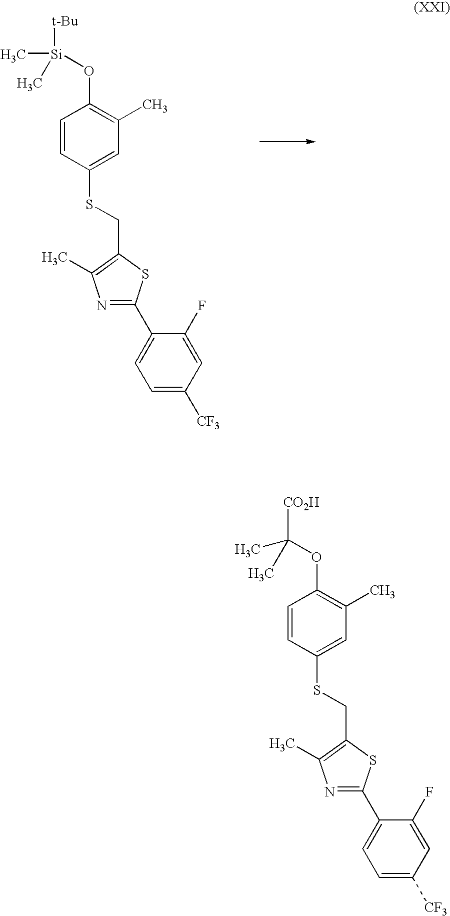

A slurry of 5-{[(4-[tert-butyl(dimethyl)silyl]oxy}-3-methylphenyl)thio]methyl}-2-[2-fluoro-4-(trifluoromethyl)phenyl]-4-methyl-1,3-thiazole (15.8 g) and sodium hydroxide (5.6 g) in acetone (120 mL) was stirred 45 min at 30° C. to effect quantitative conversion to 4-[((2-[2-fluoro-4-(trifluoromethyl)phenyl]-4-methyl-1,3-thiazol-5-yl)methyl)thio]-2-methylphenol. To the latter slurry was added dropwise over 15 min at 30-40° C. a solution of chlorotone hydrate (9.1 g) and acetone (30 mL). The resulting slurry was stirred at 35° C. for 2 h, additional chlorotone hydrate (1.9 g in 5 mL acetone) was added, and the mixture was stirred at 35° C. until ≧97% conversion was achieved. The resulting slurry was evaporated at reduced pressure to approximately one-third volume then diluted with water (100 mL) and MTBE (200 mL). To this-mixture was added 3N HCl until a pH of approximately 1 was reached. The two layers were separated and the organic solution washed with water (100 mL). The organic solution was evaporated at reduced pressure to a volume of approximately 75 mL, heptane (125 mL) was added, and the volume was reduced to approximately 75 mL. Heptane (100 mL) was added and the solution was cooled to 20° C. to effect crystallization of 2-{4-[({2-[2-fluoro-4-(trifluoromethyl)phenyl]-4-methyl-1,3-thiazol-5-yl}methyl)thio]-2-methylphenoxy}-2-methylpropanoic acid. The product was filtered, washed with heptane (3×100 mL), then dried at 55° C. in a vacuum oven to achieve a constant mass of 10.0 g (66%). The product is identical (HPLC and NMR) to that obtained by the stepwise process.

1. A process for the preparation of a compound of formula (IV), 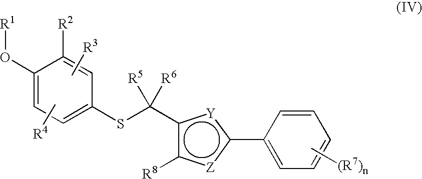
wherein,
R¹ is selected from the group consisting of H, —Si(R⁹)₃, —C(R¹⁰R¹⁰)C(O)₂H, benzyl, allyl, and C₁₋₆alkyl;
R², R³, and R⁴ are independently selected from the group consisting of H, C₁₋₃alkyl, —OCH₃, —CF₃, allyl, and halogen;
R⁵ and R⁶ are independently selected from the group consisting of H, phenyl, benzyl, C₁₋₆alkyl, and allyl;
each R⁷ is independently —CF₃, C₁₋₃alkyl, —OCH₃, or halogen;
R⁸ is selected from the group consisting of H, —CF₃, and C₁₋₆alkyl;
one of Y and Z is N and the other is S or O;
each R⁹ is independently C₁₋₆alkyl, or arylC₁₋₆alkyl, or two R⁹groups together with the silicon atom to which they are attached form a 5-7 membered ring;
each R¹⁰ is independently H or C₁₋₃alkyl, or both R¹⁰ groups together with the carbon atom to which they are attached form a 3-6 membered ring; and
n=0, 1, 2, 3, 4, or 5;
said method comprising the steps of:
a) treating of a compound of formula (I) with an alkyl lithium reagent, magnesium (0), or magnesium (0) followed by treating with a dihalo zinc (II) reagent, 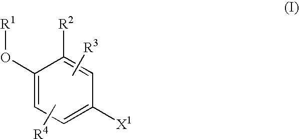
wherein,
R¹, R², R³, and R⁴ are as defined above; and
X¹ is selected from the group consisting of Cl, Br, and I;
b) followed by treating with sulfur; and
c) followed by treating with a compound of formula (III), 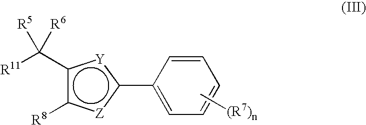
wherein,
R⁵, R⁶, R⁷, R⁸, Y, Z, and n are as defined above;
R¹¹ is Cl, Br, I, or —OS(O)₂R¹²; and
R¹² is selected from the group consisting of C₁₋₆-alkyl, C₆₋₁₀aryl, C₆₋₁₀arylC₁₋₆alkyl, and —CF₃.

2. A process according to claim 1, wherein said process is performed without isolation of intermediate compounds between steps (a) and (b) or (b) and (c).

3. A process according to claim 2, wherein
R¹ is —Si(R⁹)₃.

4. A process according to claim 2, wherein
R¹ is —Si(CH₃)₂t-Bu.

5. A process according to claim 2, wherein
R¹ is —C(R¹⁰R¹⁰)C(O)₂H.

6. A process according to claim 5, wherein R¹⁰ is —CH₃.

7. A process according to claim 2, wherein
R¹¹ is Cl or —OS(O)₂R¹², and R¹² is C₁₋₆alkyl.

8. A process according to claim 2, wherein:
R¹ is —Si(CH₃)₂t-Bu;
R² is —CH₃;
R³ and R⁴ are H;
R⁵ and R⁸ are H;
n is 2;
one R⁷ is fluorine in the ortho position and the other is —CF₃ is the para position;
R⁸ is —CH₃;
Y is S; and
Z is N.

9. A process according to claim 2, wherein:
R¹ is —C(R¹⁰R¹⁰)C(O)₂H;
R² is —CH₃;
R³ and R⁴ are H;
R⁵ and R⁶ are H;
n is 2;
one R⁷ is fluorine in the ortho position and the other is —CF₃ is the para position;
R⁸ is —CH₃;
Y is S;
Z is N; and
each R¹⁰ is —CH₃.

10. A process according to claim 8, said process further comprising the step cleaving the R¹ silyl group, to afford a compound of formula (IV), wherein R¹ is —H.

11. A process according to claim 8, said process further comprising the steps of:
d) cleaving the R¹ silyl group to afford a compound of formula (IV), wherein R¹ is —H; and
e) treating with an alkylating agent to afford a compound of formula (IV), wherein R¹ is —C(R¹⁰R¹⁰)C(O)₂H, and R¹⁰ is —CH₃.

12. A process according to claim 8, said process further comprising the steps of
d) cleaving the R¹ silyl group to afford a compound of formula (IV), wherein R¹ is —H; and
e) treating with 1,1,1-trichloro-2-methylpropan-2-ol, to afford a compound of formula (IV), wherein R¹ is —C(R¹⁰R¹⁰)C(O)₂H, and R¹⁰ is —CH₃.

13. A compound of formula (IV), 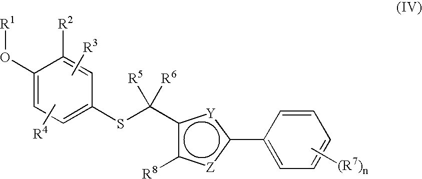
wherein:
R¹ is —Si(R⁹)₃;
R², R³, and R⁴ are independently selected from the group consisting of H, C₁₋₃alkyl, —OCH₃, —CF₃, allyl, and halogen;
R⁵ and R⁶ are independently selected from the group consisting of H, phenyl, benzyl, C₁₋₆alkyl, and allyl;
each R⁷ is independently selected from —CF₃, C₁₋₃alkyl, —OCH₃, or halogen;
R⁸ is selected from the group consisting of H, —CF₃, and C₁₋₆alkyl;
one of Y and Z is N and the other is S or O;
each R⁹ is independently selected from C₁₋₆alkyl, arylC₁₋₆alkyl, or two R⁹ groups together with the silicon atom to which they are attached form a 5-7 membered ring; and
n=0, 1, 2, 3, 4, or 5.

14. A compound according to claim 13, wherein:
R¹ is —Si(R⁹)₃;
R² is —CH₃;
R³, R⁴, R⁵, and R⁶ are hydrogen;
n is 2;
one R⁷ is F in the ortho position and the other is —CF₃ in the para position;
R⁸ is —CH₃;
R⁹ is C₁₋₆alkyl;
Y is S; and
Z is N.

15. A compound according to claim 14, wherein R¹ is —Si(CH₃)₂t-Bu.

16. A compound of formula (V), 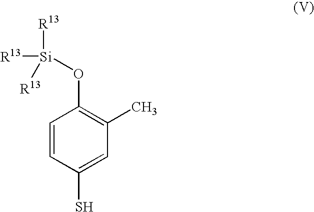
wherein:
R¹³ is C₁₋₆alkyl, C₆₋₁₄arylC₁₋₆alkyl, or C₆₋₁₄aryl.

17. A compound according to claim 16, wherein two R¹³ are —CH₃ and the other is t-Bu.

18. A process for the preparation of compounds of formula (III), 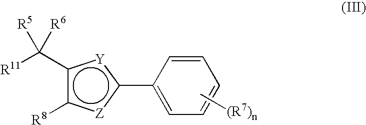
wherein:
R⁵ and R⁶ are independently selected from the group consisting of H, phenyl, benzyl, C₁₋₆alkyl, and allyl;
each R⁷ is independently selected from —CF₃, C₁₋₃alkyl, —OCH₃, or halogen;
R⁸ is H, —CF₃, or C₁₋₆alkyl;
one of Y and Z is N and the other is S or O;
R¹¹ is —OH; and
n=0, 1, 2, 3, 4, or 5;
said process comprising the step of treating a compound of formula (XVII) with thioacetic acid, 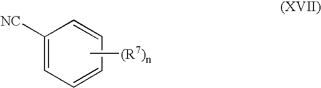
wherein:
each R⁷ is independently selected from —CF₃, C₁₋₃alkyl, —OCH₃, or halogen; and
n=0, 1, 2, 3, 4, or 5.

19. A process according to claim 18, wherein said process further comprises the step of treating with an □-halo-□-ketoester.

20. A process according to claim 19, wherein said process further comprises the step of treating with a reducing agent.

21. A process according to claim 20, wherein R⁵ and R⁶ are hydrogen, n is 2, one R⁷ is fluorine and the other is —CF₃, R⁸ is C₁₋₆alkyl, Y is S, Z is N, and R¹¹ is —OH.

22. A process according to claim 21, wherein one R⁷ is fluorine in the ortho position and the other is —CF₃ in the para position, and R⁸ is —CH₃.

23. A process according to claim 20, wherein the compound of formula (III) is {2-[2-fluoro-4-(trifluoromethyl)phenyl]-4-methyl-1,3-thiazol-5-yl}methanol.

24. (canceled)
